(19) United States
(12) Patent Application Publication
(10) Pub. No.: US 2010/0217367 A1
(43) Pub. Date: Aug. 26, 2010

(54) TRANSESOPHAGEAL IMPLANTATION OF CARDIAC ELECTRODES AND DELIVERY OF CARDIAC THERAPIES

(76) Inventor: Amir Belson, Los Altos, CA (US)

Correspondence Address: GREGORY SMITH & ASSOCIATES, 3900 NEWPARK MALL ROAD, 3RD FLOOR, NEWARK, CA, 94560, US

(21) Appl. No.: 12/520,984

(22) PCT Filed: Jun. 25, 2007
(86) PCT No.: PCT/US07/14715

§ 371 Date: Apr. 19, 2010

Related U.S. Application Data
(60) Provisional application No. 60/816,160, filed on Jun. 23, 2006.
(60) Provisional application No. 60/855,253, filed on Oct. 30, 2006.

(51) Int. Cl. A61N 1/05

(52) U.S. Cl. 607/119

(57) ABSTRACT

The present invention provides for transesophageal implantation of cardiac electrodes (102) and (104) for pacing and/or defibrillation The electronic module (110) could be implanted in the abdomen or thorax using a transesophageal, transgastric, thoracoscopic or open surgical approach In another aspect, the invention provides for transesophageal delivery of other cardiac therapies including ablation, phototherapy, radiation therapy and implantation or injection of therapeutic substances into the heart The transesophageal approach takes advantage of the proximity between the esophagus and the chambers of the heart, allowing surgical access to the heart with very low morbidity and risk of complications.

### CROSS REFERENCE TO OTHER APPLICATIONS

This application claims the benefit of U.S. Provisional Application 60/______, TRANSESOPHAGEAL IMPLANTATION OF CARDIAC ELECTRODES, filed on Jun. 26, 2006 and U.S. Provisional Application 60/______, TRANSESOPHAGEAL DELIVERY OF CARDIAC THERAPIES, filed on Oct. 30, 2006. These and all patents and patent applications referred to herein are hereby incorporated by reference.

### FIELD OF THE INVENTION

This invention relates generally to implantable electrodes for cardiac pacing, defibrillation, ablation and/or therapy delivery. More specifically, it relates to methods and devices for implanting cardiac electrodes and/or therapy delivery using a transesophageal surgical approach.

### BACKGROUND OF THE INVENTION

Implantable cardiac electrophysiology devices, such as cardiac pacemakers and implantable cardiac defibrillators, generally require one or more electrodes to be implanted in or near a chamber of the heart for delivering electrical signals to the tissue to be stimulated. Currently cardiac electrodes are implanted surgically using an open chest or thoracoscopic approach or, more frequently, by catheter techniques using a transvenous approach. The surgical approach allows optimization of electrode placement, but it is associated with a higher degree of morbidity and mortality. Transvenous electrodes are effective, but they are also associated with complications, such as infection, venous thrombosis and electrode dislodgement.

Temporary pacing or emergency defibrillation can be accomplished using external electrodes. However, a relatively high current must be applied to stimulate the heart because only a fraction of the current applied will actually pass through the heart. Percutaneous cardiac stimulation devices have been devised for making direct electrode contact with the epicardium through an intercostal puncture or incision. (e.g. Zadini et al U.S. Pat. No. 5,978,714 Epicardial percutaneous device for electrical cardiac stimulation.) This approach allows a much lower pacing or defibrillation current to be used, but there is significant risk of morbidity and complications associated with making a puncture or incision into the chest. Temporary transesophageal pacing or defibrillation probes provide a less invasive approach that takes advantage of the proximity between the esophagus and the chambers of the heart allowing a much lower pacing or defibrillation current to be used. (e.g. Pless et al U.S. Pat. No. 4,640,298 Esophageal electrode probe useful for electrical stimulation heart; Cohen U.S. Pat. No. 5,417,713 Tranesophageal defibrillating system.) Mesallum (U.S. Pat. No. 6,689,062) describes a method and apparatus for transesophageal cardiovascular procedures.

### SUMMARY OF THE INVENTION

In one aspect, the present invention provides for transesophageal implantation of cardiac electrodes for pacing and/or defibrillation. The transesophageal approach takes advantage of the proximity between the esophagus and the chambers of the heart, allowing surgical access to the heart with very low morbidity and risk of complications. The transesophageal surgical approach allows optimization of electrode placement while avoiding many of the potential complications of the standard surgical and transvenous approaches.

An esophageal access device with a working channel, such as an access sheath, flexible endo scope or transesophageal echography probe, is inserted into the patient's esophagus. A flexible shaft needle or trocar is inserted through the working channel to puncture the esophageal wall in the vicinity of the patient's heart. The needle or trocar is withdrawn and one or more electrode wires are inserted through the incision in the esophagus and implanted in the selected chambers of the heart to provide optimal pacing and/or defibrillation. The placement of the electrodes can be tested at this point by measuring the pacing and/or defibrillation threshold currents. Once satisfactory electrode placement has been obtained, the electronic module of the pacemaker and/or defibrillator, sometimes referred to as the “can”, is then implanted and connected to the electrodes.

One approach is to implant the electronic module in the patient's abdomen and to run the electrode wires internally through the esophagus into the stomach. Using a flexible endoscope, a puncture can be made in the gastric wall to feed the electrode wires through to the electronic module.

Another approach is to implant the electronic module in the patient's abdomen and to run the electrode wires externally along the outside of the esophagus and through the diaphragm into the abdomen. A laparoscopic, transgastric or open surgical approach can be used to connect the electrode wires to the electronic module within the abdomen.

Alternatively, the electronic module can be implanted within the patient's thorax, for example in a subxiphoid or intercostal position. The electrode wires can be run internally or externally along the esophagus and a thoracoscopic or open surgical approach can be used to connect the electrode wires to the electronic module.

In another aspect, the present invention provides for transesophageal delivery of other cardiac therapies including ablation, phototherapy, radiation therapy and implantation or injection of therapeutic substances into the heart.

### BRIEF DESCRIPTION OF THE DRAWINGS

FIG. 1 shows an internal lateral view of a patient with an esophageal access device inserted into the esophagus and two electrodes attached to the patient's heart.

FIG. 2 shows an internal lateral view of a patient with the electrodes connected to a pacemaker and/or defibrillator implanted in the abdomen. The electrode wires are connected via a transesophageal and transgastric route.

FIG. 3 shows an internal lateral view of a patient with the electrodes connected to a pacemaker and/or defibrillator implanted in the abdomen. The electrode wires pass external to the esophagus and into the abdomen through the esophageal hiatus in the diaphragm.

FIG. 4 shows a flexible, small diameter embodiment of an electronic module for a pacemaker and/or defibrillator.

FIG. 5 shows a flexible, coil-shaped embodiment of an electronic module for a pacemaker and/or defibrillator.

FIG. 6 shows a corkscrew-shaped electrode implanted into the myocardium.

FIG. 7 shows a corkscrew-shaped electrode implanted into the myocardium with a helical strain relief that reduces bending stress and spaces the pericardium away from the epicardial surface.

FIG. 8 shows a balloon device that can be used to create a space between the pericardium and the epicardial surface to facilitate electrode placement.

FIG. 9 is a schematic diagram of a magnetic navigation system that can be used to assist in electrode placement.

FIG. 10 illustrates a method of electrophysiological mapping of the heart using the magnetic navigation system or other means.

### DESCRIPTION OF THE INVENTION

The present invention provides methods and devices for transesophageal implantation of cardiac electrodes for pacing and/or defibrillation. An esophageal access device with a working channel is inserted into the patient's esophagus. The access device can be a tubular access sheath, or a flexible endoscope or transesophageal echography probe with a working channel. Advantageously, the working channel will have a lateral exit port near the distal end of the device so that instruments can be directed toward the esophageal wall. Alternatively, the exit port will be located at the distal end with an ability to change the position and/or curvature of the distal part of the endoscope or tubular structure. The position of the access device with respect to the heart can be monitored using transesophageal echography (TEE), transthoracic ultrasound imaging and/or fluoroscopy. A tubular access sheath or a flexible endoscope can be used in conjunction with a TEE probe, such as a nonocclusive transesophageal echography probe as described in a copending patent application entitled DETACHABLE TRANSESOPHAGEAL ULTRASOUND IMAGING DEVICE by the same inventor.

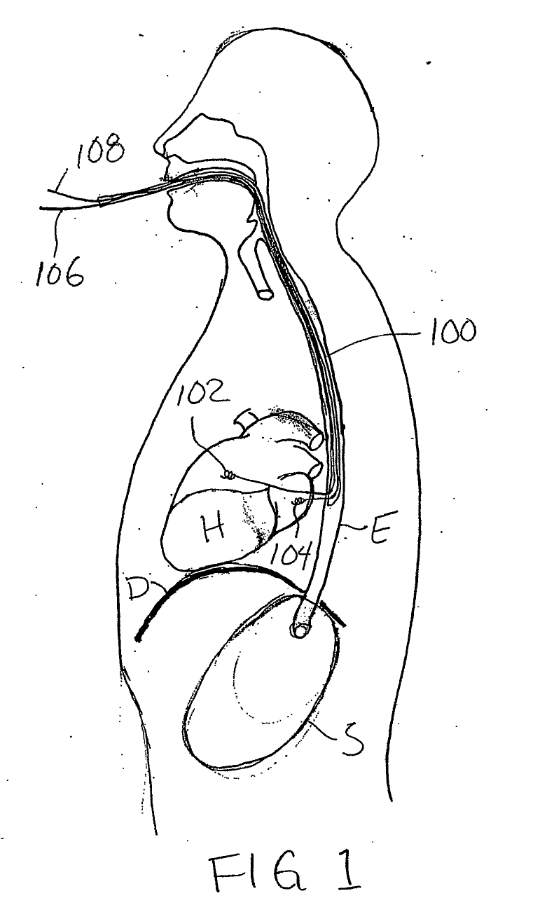

The esophageal access device is maneuvered so that the exit port is in the vicinity of the patient's heart, then a flexible shaft needle or trocar is inserted through the working channel to puncture the esophageal wall. Alternatively, the esophageal puncturing device and treatment apparatus can be part of the endoscope or access sheath tip or integrated into an esophageal probe with or without a working channel. The needle or trocar is withdrawn and one or more electrode wires are inserted through the incision in the esophagus and implanted in the selected chambers of the heart using an electrode insertion device to provide optimal pacing and/or defibrillation. Optionally, an electrode insertion device can be integrated with a needle or trocar to facilitate insertion of the electrodes with a minimum of manipulation and instrument changes. In one optional configuration the electrode insertion device can be integrated with a shape-controlled cannula or catheter that can puncture the esophageal wall and deliver the electrodes to the desired position in a very efficient manner. A mechanized electrode insertion device can further facilitate electrode insertion. For example, an air pressure actuated device can be used to insert barbed electrodes into the myocardium. FIG. 1 shows an internal lateral view of a patient with an esophageal access device 100 inserted into the esophagus E and two electrodes 102, 104 attached to the patient's heart H. By way of example, the electrodes are shown implanted into the patient's left and right atria. The ventricles of the heart are also accessible by this method, for example for ventricular or atrioventricular pacing.

The placement of the electrodes can be tested at this point by measuring the pacing and/or defibrillation threshold currents. If the intention is to provide temporary pacing and/or emergency defibrillation, the electrode wires, 106, 108, which extend out the patient's mouth, can be connected to an external pacemaker and/or defibrillator to perform the necessary procedures. Alternatively, an electronic module could be temporarily implanted within the patient's esophagus. After the procedures have been performed, the electrodes and/or electronic module can be removed if they are no longer needed.

If the intention is to provide long-term pacing and/or defibrillation, the electronic module of the pacemaker and/or defibrillator, sometimes referred to as the “can”, is implanted and connected to the electrodes.

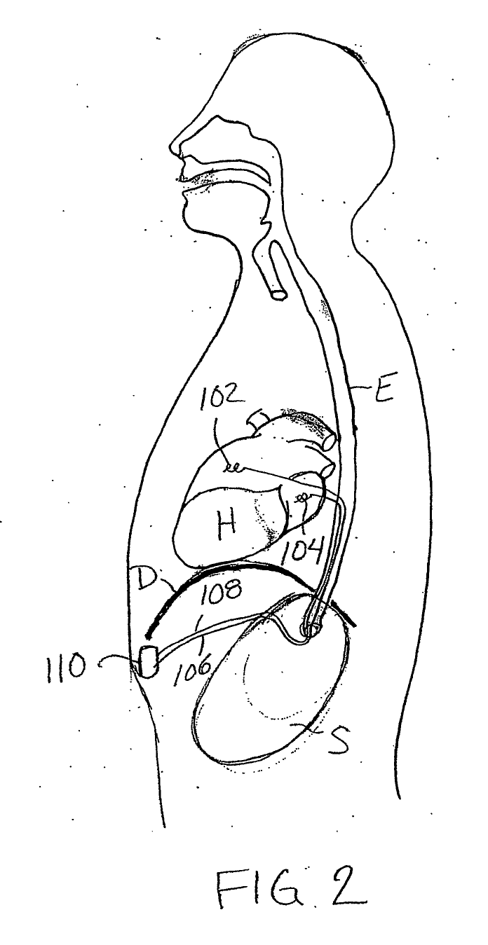

One approach, which is illustrated in FIG. 2, is to implant the electronic module 110 in the patient's abdomen and to run the electrode wires internally through the esophagus E into the stomach S. Using a flexible endoscope, a puncture can be made in the gastric wall to feed the electrode wires 106, 108 through to the electronic module 110. Placement and connection of the electrode wires can be assisted using transgastric, laparoscopic and/or open surgical techniques.

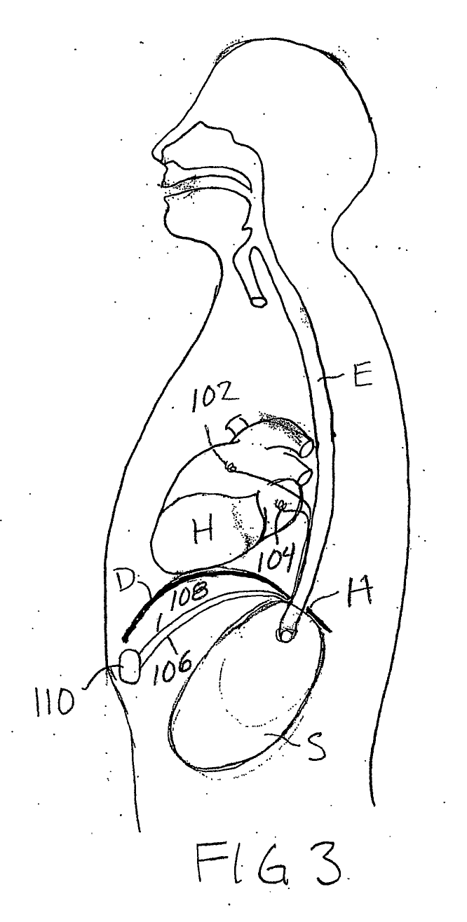

Another approach, which is illustrated in FIG. 3, is to implant the electronic module in the patient's abdomen and to run the electrode wires 106, 108 externally along the outside of the esophagus E and through the esophageal hiatus H, an opening in the diaphragm D, and into the abdomen. A laparoscopic, transgastric or surgical approach can be used to connect the electrode wires to the electronic module within the abdomen.

The electronic module can be implanted within the patient's abdomen in a subxiphoid position or just below the ribs or it can be anchored to the linea alba, a very fibrous membrane within the abdomen. Alternatively, the electronic module can be implanted within the patient's thorax, for example in a subxiphoid or intercostal position. A subxiphoid or intercostal incision can be used to insert the electronic module into the thorax just inside of the ribcage. The electrode wires can be run internally or externally along the esophagus and a thoracoscopic or open surgical approach can be used to connect the electrode wires to the electronic module.

Another, less invasive, approach would be to implant electrodes internal or external to the esophagus rather than directly in the myocardium. The electrodes could be used effectively for sensing, pacing and/or defibrillation with only slightly higher current than directly implanted electrodes. The electronic module could be implanted in the abdomen or thorax using a transesophageal, transgastric, thoracoscopic or open surgical approach. If it is intended for temporary use, the electronic module could be temporarily implanted within the patient's esophagus.

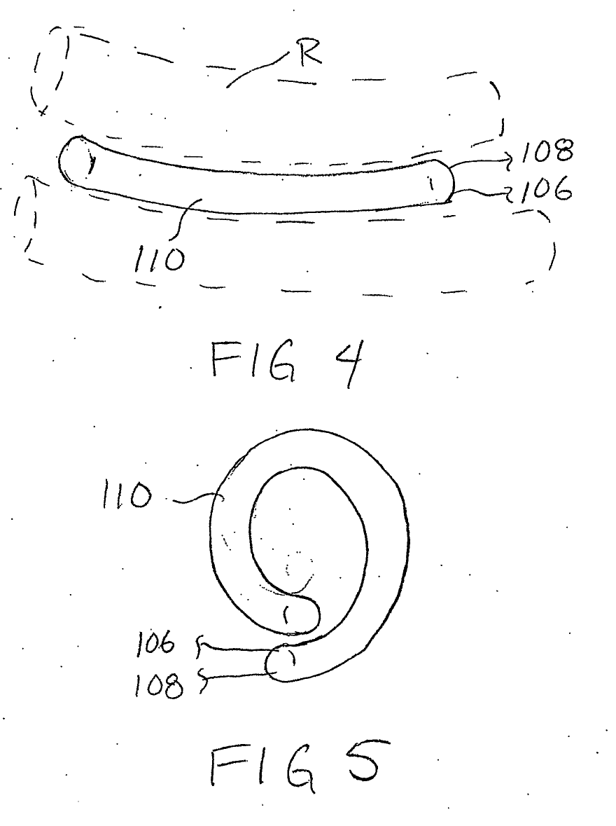

Preferably, the electronic module will be configured so that it will be comfortable and unobtrusive to the patient wherever it is implanted. In one preferred embodiment shown in FIG. 4, the electronic module 110 is in an elongated, flexible, small diameter configuration. This configuration lends itself to unobtrusive implantation into an intercostal space, just below the ribs R or various places within the abdomen using a very small incision. FIG. 5 shows another embodiment of an electronic module 110 in a flexible, coil-shaped configuration. This configuration lends itself to implantation into the abdominal space using a very small incision. The electronic module assumes a flat, coiled configuration after insertion, which would be very unobtrusive to the patient on the anterior wall of the abdomen. Alternatively, the electronic module could be a compact “can” shape as used with existing pacemakers and implantable defibrillators.

Optionally, the electronic module can have an external electrode to act as a ground electrode for the pacemaker and/or defibrillator function of the device. Another optional feature of the electronic module would be to provide transcutaneous recharging of the batteries, which would reduce the size of the batteries needed in the implantable device and prolong the useful life of the device. Optionally, the electronic module can be implanted inside of a sack of a material that resists tissue ingrowth or even a sack within a sack to reduce the likelihood of adhesions to the electronic module so that it can be repaired or replaced if ever needed in the future.

The cardiac electrodes can be attached or anchored to the heart using known techniques adapted from transvenous, transthoracic or surgically implanted electrode technologies. FIG. 6 shows a corkscrew-shaped electrode 112 that is particularly well adapted for implantation into the myocardium using the transesophageal approach. The electrode is inserted through the working channel of the access device and then through the puncture in the esophageal wall to the heart using a flexible electrode insertion device. Using the insertion device, the corkscrew-shaped electrode is pressed against the pericardium P and rotated to screw it through the pericardium P and into the epicardial surface of the heart H. Then, the insertion device is withdrawn. The electrode insertion device may have a braided tubular construction that functions as a flexible torque transmission shaft.

FIG. 7 shows a corkscrew-shaped electrode 102 implanted into the myocardium with a helical strain relief 112 that reduces bending stress and spaces the pericardium P away from the epicardial surface of the heart H. The implantation of this electrode is similar to the corkscrew-shaped electrode of FIG. 6, except that is done in two steps. First, the corkscrew-shaped electrode 102 is screwed into the pericardium while pulling back a little to tent the pericardium. If desired, a flexible, endoscopic grasper or a suction grasper can be used to facilitate this step. Once the corkscrew-shaped electrode 102 is through the pericardium, the corkscrew-shaped electrode is screwed into the epicardium. The helical strain relief 112 rotates through the pericardium and maintains the spacing between the pericardium and the epicardial surface to reduce the likelihood of adhesions. The helical strain relief 112 also reduces bending stress in the electrode wire 106 to reduce the likelihood of fatigue or bending failure of the electrode wire.

FIG. 8 shows a balloon device 114 that can be used to create a space between the pericardium P and the epicardial surface of the heart H to facilitate electrode placement. The balloon device 114 can be used during implantation of either of the electrodes of FIGS. 6 and 7 or with other known electrode configurations. The balloon device 114 can be deployed beside the electrode 102 during implantation or, alternatively, the balloon device can be configured with a through lumen 116 to allow the electrode to be deployed through the balloon device. After implantation of the electrode(s), the balloon device is deflated and withdrawn. The balloon device creates a space between the pericardium and the epicardial surface to reduce the likelihood of adhesions.

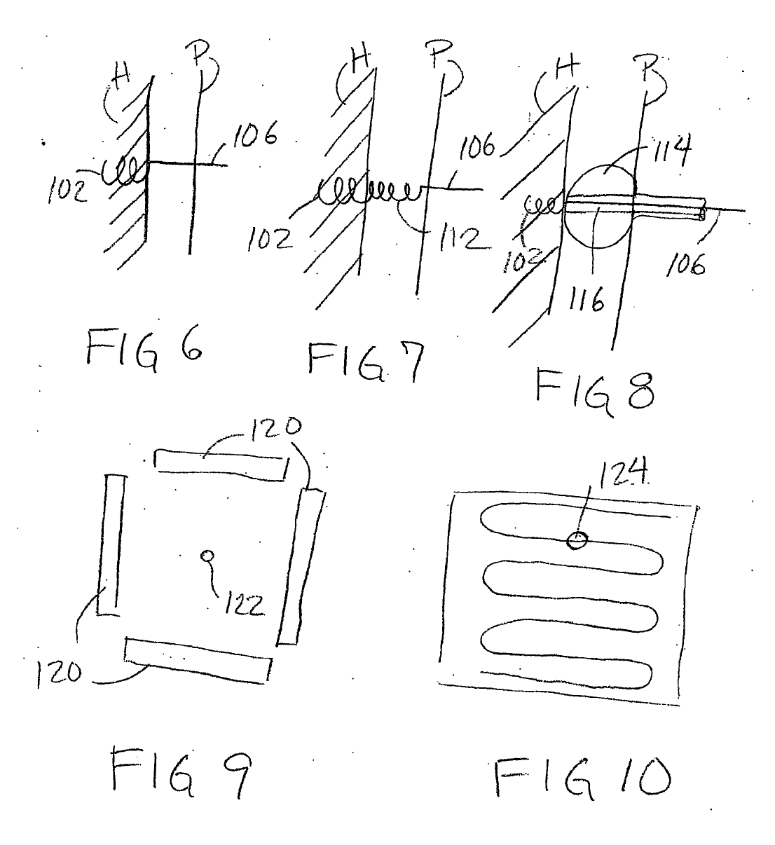

FIG. 9 is a schematic diagram of a magnetic navigation system that can optionally be used to assist in electrode placement. Strong electromagnets 120 are positioned external to the patient to create a magnetic field in three dimensions. A magnet or a magnetically attracted material 122 is mounted near the distal tip of the electrode insertion device. By manipulating the electrode insertion device and varying the X, Y and Z components of the three-dimensional magnetic field, the magnetic tip 122 of the electrode insertion device can be steered to a desired location on the epicardium.

One useful application of the magnetic navigation system of FIG. 9 is mapping the epicardial surface to find the optimum positions for the pacing and/or defibrillation electrodes. FIG. 10 illustrates a method of electrophysiological mapping of the heart where a surface electrode 124 is slid along a serpentine path on the epicardial surface using the magnetic navigation system described above in order to find the optimum electrode placement. Once the desired positions are established, a permanent electrode, such as those described above, can be used to replace the surface electrode. Alternatively, other patterns of movement may be used to map the epicardial surface and other means, such as a mechanical device, can be used to effect the movement of the surface electrode.

Another application of the transesophageal approach described herein is to deliver an ablation device or other therapeutic device to the heart through the esophageal wall. For example, an ablation catheter or band can be introduced in the vicinity of the pulmonary veins to create a circular lesion around the pulmonary veins to treat atrial fibrillation or other arrhythmias. One option would be to deliver an ablation device or devices through the esophageal wall from above and below the pulmonary veins to facilitate creating a full circular lesion around the pulmonary veins.

Other therapies can be performed using the transesophageal approach described herein. For example, a catheter or probe with a light source can be inserted using the transesophageal approach for phototherapy on the heart or other structures within the chest and mediastinum. One or more optical fibers can be used to deliver light through the catheter to the target site from a light source located external to the patient. Alternatively, a light source such as one or more light emitting diodes or diode lasers can be mounted on a distal region of a catheter or probe. A power supply for the light source can be located external to the patient or a battery and any necessary circuitry can be integrated into the catheter or probe. Preferably, the device will include a power switch located near a proximal end of the catheter or probe for selectively activating the light source. Optionally, a timer may be included to time the duration of the phototherapy treatment and/or to maintain the light exposure within predetermined safety limits. Optionally, the light source may include a reflector or the like to direct the light in a desired direction and to shield other surrounding structures from the effects of the phototherapy. The light source can be used to deliver phototherapy to the target tissues on an acute basis for minutes, hours or days, after which the catheter or probe can be withdrawn.

Alternatively, an implantable light source can be used to deliver chronic phototherapy. An implantable light source that includes one or more light emitting diodes or diode lasers, a battery and any necessary circuitry can be inserted via the transesophageal approach using a catheter or probe. The implantable light source may include hooks, barbs, sutures, adhesives or other means for attaching the light source at a desired position within the anatomy. One or more implantable light sources can be implanted within the pericardium, on the epicardial surface, or external to the pericardium. Optionally, the implantable light source may include a reflector or the like to direct the light in a desired direction and to shield other surrounding structures from the effects of the phototherapy. The implantable light source can deliver phototherapy to the target tissues for days, weeks or even months. After completion of the phototherapy regimen, the implantable light sources can be retrieved or they can simply be left in place as long as their presence does not cause undesired effects. Optionally, a thin tether or other unobtrusive structure attached to the implantable light sources can be left in place to facilitate retrieval.

Optionally, the implantable light source may include a remotely controllable power switch for selectively activating the light source. The power switch can be configured to turn on and off the light source using a variety of different remote control means, such as a radio signal, a change in magnetic field, etc. Alternatively, the implantable light source can be activated to turn on the light source prior to implantation and simply left to run until the power supply is exhausted or the device is removed. Alternatively, the implantable light source can be remotely activated through the catheter or probe or activated by withdrawal of the catheter or probe from the light source at the appropriate time during the implantation procedure. Whether it is manually activated or remotely controlled, the device may also include a timer to time the duration of the phototherapy treatment and/or to maintain the light exposure within predetermined safety limits.

Similarly, the transesophageal approach can be used to deliver a radiation source for brachytherapy of the heart or other structures within the chest and mediastinum. For example, a catheter or probe with a radiation source attached can be inserted via the transesophageal approach for acute brachytherapy. Alternatively, an implantable radiation source can be temporarily or permanently implanted using a catheter or probe inserted via the transesophageal approach for chronic brachytherapy. A tether or similar structure can be attached to the implantable radiation source to facilitate later removal. In either case, the radiation source can be a passive source, such as a radioactive material (e.g. Strontium-90 or Iridium-192), and/or an active source, such as an electron beam or X-ray source, that can be selectively activated to emit radiation.

Passive radiation sources that use a radioactive material will preferably be covered by a radiation shield or capsule of radiopaque material prior to and during insertion to protect medical personnel and tissues that are not the intended target tissue from unintended radiation exposure. The radiation shield or capsule can be opened and/or removed from the radiation source to expose the target tissue to the intended dose of radiation. Whether using a passive or active radiation source, a radiation shield can also be used to provide directionality and to protect surrounding tissue from unintended radiation exposure.

Optionally, the implantable radiation source may include a remote control means to open and close the radiation shield or capsule and/or to switch on and off the power to an active radiation source. The remote control means can operate using a radio signal, a change in magnetic field, etc. Alternatively, the implantable radiation source can be remotely activated through the catheter or probe or activated by withdrawal of the catheter or probe from the radiation source at the appropriate time during the implantation procedure. Whether it is manually activated or remotely controlled, the device may also include a timer to time the duration of the radiation treatment and/or to maintain the radiation exposure within predetermined safety limits.

Other therapies that can be performed using the transesophageal approach involve the release of therapeutic substances into the heart or other structures within the chest and mediastinum. Using a catheter or probe, therapeutic substances can be placed within the pericardium to be absorbed through the epicardial surface and/or injected directly into the myocardium. The therapeutic substances can be injected or released as a single dose and/or they can be injected or released slowly over an extended period of time. Slow release can be accomplished by coating or encapsulating the therapeutic substance with a dissolvable or absorbable coating or by implanting a pressurized reservoir or a reservoir connected to a pump that releases or injects the therapeutic substance slowly over an extended period of time. Optionally, a catheter and/or needle can be connected to the reservoir to inject the therapeutic substances into the heart or other tissues. Optionally, energy from the contraction of the myocardium can be used to pressurize the reservoir.

The therapeutic substances can include substances to encourage or inhibit the growth of blood vessels (angiogenesis) and/or substances to encourage or inhibit the growth of cardiac muscle tissue (myogenesis). Another class of therapeutic substances would include stem cells or other differentiated or non-differentiated progenitor cells that could be injected into the tissue, for example to treat cardiomyopathy or congestive heart failure. Alternatively or in addition, drugs, such as steroids and antibiotics can also be released or injected.

The transesophageal approach would have certain advantages over current methods of delivering therapeutic substances to the heart. It would be much less invasive than open-chest or thoracoscopic surgical approaches, more on the level of current catheter-based intravascular approaches in terms of invasiveness. However, there would not be the problems of washout of the therapeutic substances and unintended downstream or systemic exposure to the therapeutic substances that are inherent with the intravascular approach.

Phototherapy, radiation treatment and release or injection of therapeutic substances via a transesophageal approach can be used separately or in combination in the treatment of many different conditions. Examples include, but are not limited to, atherosclerosis, restenosis, arrhythmias, cardiomyopathy, hypertrophic cardiomyopathy, congestive heart failure, tumors of the myocardium, etc.

While the present invention has been described herein with respect to the exemplary embodiments and the best mode for practicing the invention, it will be apparent to one of ordinary skill in the art that many modifications, improvements and subcombinations of the various embodiments, adaptations and variations can be made to the invention without departing from the spirit and scope thereof.

1. Apparatus for implanting a cardiac electrophysiology device in a patient, comprising:
an esophageal access device with a working channel extending therethrough;
incision means extendable through the working channel of the esophageal access device for making an incision through the patient's esophageal wall in the vicinity of the patient's heart;
at least one electrode configured for introduction through the incision in the esophageal wall and placement in electrical contact with the patient's heart;
an electrophysiology device electronic module configured for implantation within a cavity in the patient's body; and
at least one electrode wire for connecting the electrode to the electrophysiology device electronic module.

2. The apparatus of claim 1, wherein the electrophysiology device electronic module has an elongated, flexible, small diameter configuration, permitting implantation into an intercostal space.

3. The apparatus of claim 1, wherein the electrophysiology device electronic module has a flexible, coil-shaped configuration, permitting implantation into an abdominal space through a very small incision.

4. The apparatus of claim 1, further comprising:
an inflatable balloon configured for maintaining a space between the patient's pericardium and epicardial surface during implantation of the electrode to reduce the likelihood of adhesions.

5. The apparatus of claim 1, wherein the electrode comprises a helical strain relief configured for maintaining a space between the patient's pericardium and epicardial surface to reduce the likelihood of adhesions after implantation of the electrode.

6. The apparatus of claim 1, wherein the electrophysiology device electronic module is a cardiac defibrillator electronic module.

7. The apparatus of claim 1, wherein the electrophysiology device electronic module is a cardiac pacemaker electronic module.

8. The apparatus of claim 1, further comprising:
an electrode insertion device having a magnet or a magnetically attracted material mounted near a distal tip of the electrode insertion device.

9. The apparatus of claim 8, further comprising:
at least one external magnet for directing movement of the magnet or magnetically attracted material to navigate the distal tip of the electrode insertion device within the patient's body.

10. The apparatus of claim 8, further comprising:
electromagnets positioned external to the patient to create a controllable three-dimensional magnetic field for directing movement of the magnet or magnetically attracted material to navigate the distal tip of the electrode insertion device within the patient's body.

11. A method of implanting a cardiac electrophysiology device in a patient, comprising:
making an incision through the patient's esophageal wall in the vicinity of the patient's heart;
introducing an electrode through the incision in the esophageal wall;
placing the electrode in electrical contact with the patient's heart;
implanting an electrophysiology device electronic module within a cavity in the patient's body; and
connecting an electrode wire from the electrode to the electrophysiology device electronic module.

12. The method of claim 11, wherein the electrophysiology device electronic module is a cardiac defibrillator electronic module.

13. The method of claim 11, wherein the electrophysiology device electronic module is a cardiac pacemaker electronic module.

14. The method of claim 11, wherein the electrode wire from the electrode to the electrophysiology device electronic module passes through a lumen of the patient's esophagus.

15. The method of claim 11, wherein the electrode wire from the electrode to the electrophysiology device electronic module passes external to the patient's esophagus.

16. The method of claim 11, wherein the electrophysiology device electronic module is implanted in a subxiphoid position.

17. The method of claim 11, wherein the electrophysiology device electronic module is implanted in an intercostal position.

18. The method of claim 11, wherein the electrophysiology device electronic module is implanted through an incision in the patient's gastric wall.

19. A method of treating a patient, comprising:
making an incision through the patient's esophageal wall in the vicinity of the patient's thorax;
introducing a radiation emitting element into the patient's thorax through the incision in the esophageal wall; and
emitting radiation from the radiation emitting element into the patient's thorax.

20. (canceled)

21. (canceled)

22. (canceled)

23. (canceled)

24. A method of treating a patient, comprising:
making an incision through the patient's esophageal wall in the vicinity of the patient's thorax;
introducing a therapeutic substance into the patient's thorax through the incision in the esophageal wall; and
closing the incision in the esophageal wall.

25. (canceled)

26. (canceled)

27. (canceled)

28. (canceled)

29. (canceled)

30. (canceled)

31. (canceled)

32. (canceled)

33. (canceled)
